(19) United States
(12) Patent Application Publication
(10) Pub. No.: US 2019/0069757 A1
(43) Pub. Date: Mar. 7, 2019

(54) ENDOSCOPIC IMAGE PROCESSING APPARATUS

(71) Applicant: OLYMPUS CORPORATION, Tokyo (JP)

(72) Inventor: Hidekazu IWAKI, Tokyo (JP)

(73) Assignee: OLYMPUS CORPORATION, Tokyo (JP)

(21) Appl. No.: 16/180,304

(22) Filed: Nov. 5, 2018

Related U.S. Application Data
(63) Continuation of application No. PCT/JP2016/065137, filed on May 23, 2016.

(51) Int. Cl. A61B 1/00; G06T 7/20; G06T 7/70

(52) U.S. Cl. CPC A61B 1/0005; G06T 7/20; G06T 2207/20172; G06T 2207/10068; G06T 7/70

(57) ABSTRACT

An endoscopic image processing apparatus includes a processor, and the processor performs processing for detecting a region-of-interest from sequentially inputted observation images; performs enhancement processing of a position corresponding to the region-of-interest, on the observation images of the subject inputted after a first period elapses from a time point of a start of detection of the region-of-interest, when the region-of-interest is continuously detected; and sets the first period based on at least one of position information indicating a position of the region-of-interest in the observation images and size information indicating a size of the region-of-interest in the observation images.

### CROSS REFERENCE TO RELATED APPLICATION

This application is a continuation application of PCT/JP2016/065137 filed on May 23, 2016, the entire contents of which are incorporated herein by this reference.

### BACKGROUND OF INVENTION

### 1. Field of the Invention

The present invention relates to an endoscopic image processing apparatus.

### 2. Description of the Related Art

Conventionally, in an observation using an endoscope apparatus, an operator determines a presence or absence of a lesion part by viewing an observation image In order to prevent an operator from overlooking a lesion part when viewing an observation image, an endoscope apparatus configured to display an observation image, with an alert image being added to a region-of-interest detected by image processing is proposed as disclosed in Japanese Patent Application Laid-Open Publication No. 2011-255006, for example.

### SUMMARY OF THE INVENTION

An endoscopic image processing apparatus according to one aspect of the present invention includes a processor. The processor performs processing for detecting a region-of-interest from sequentially inputted observation images of a subject, performs enhancement processing of a position corresponding to the region-of-interest, on the observation images of the subject inputted after a first period elapses from a time point of a start of detection of the region-of-interest, when the region-of-interest is continuously detected, and sets the first period based on at least one of position information indicating a position of the region-of-interest in the observation images and size information indicating a size of the region-of-interest in the observation images.

### BRIEF DESCRIPTION OF THE DRAWINGS

FIG. 1 is a block diagram showing a schematic configuration of an endoscope system including an endoscopic image processing apparatus according to an embodiment of the present invention.

FIG. 2 is a block diagram showing a configuration of a detection support section of the endoscope system according to the embodiment of the present invention.

FIG. 3 is an explanatory diagram illustrative of an example of a screen configuration of an image for display of the endoscope system according to the embodiment of the present invention.

FIG. 4 is a flowchart describing one example of processing performed in the endoscope system according to the embodiment of the present invention.

FIG. 5 is a flowchart describing one example of processing performed in the endoscope system according to the embodiment of the present invention.

FIG. 6 is a schematic diagram showing one example of a classifying method of respective parts of an observation image to be used in the processing in FIG. 5.

FIG. 7 is a view illustrating one example of a screen transition of an image for display in accordance with the processing performed in the endoscope system according to the embodiment of the present invention.

### DETAILED DESCRIPTION OF THE PREFERRED EMBODIMENT(S)

Hereinafter, embodiments of the present invention will be described with reference to drawings.

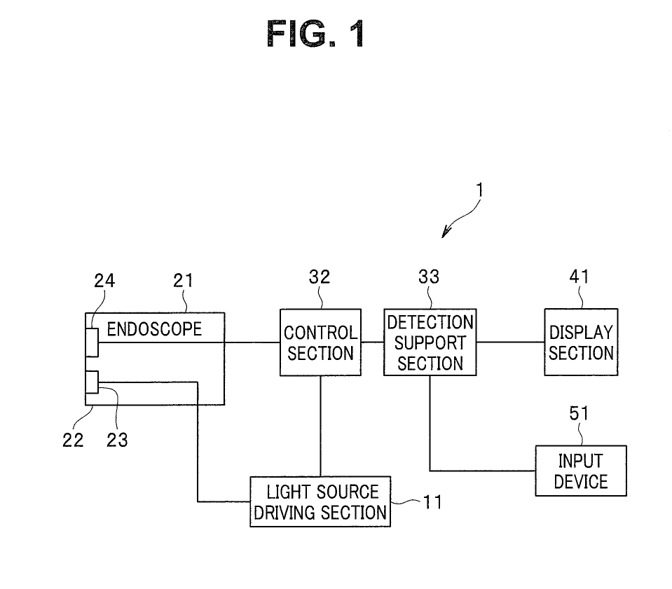

FIG. 1 is a block diagram showing a schematic configuration of an endoscope system including an endoscopic image processing apparatus according to an embodiment of the present invention.

The schematic configuration of an endoscope system 1 will be described. The endoscope system 1 includes a light source driving section 11, an endoscope 21, a control section 32, a detection support section 33, a display section 41, and an input device 51. The light source driving section 11 is connected to the endoscope 21 and the control section 32. The endoscope 21 is connected to the control section 32. The control section 32 is connected to the detection support section 33. The detection support section 33 is connected to the display section 41 and the input device 51. Note that the control section 32 and the detection support section 33 may be configured as separate devices, or may be provided in the same device.

The light source driving section 11 is a circuit configured to drive an LED 23 provided at a distal end of an insertion portion 22 of the endoscope 21. The light source driving section 11 is connected to the control section 32 and the LED 23 in the endoscope 21. The light source driving section 11 receives a control signal from the control section 32, to output a driving signal to the LED 23, to thereby be capable of driving the LED 23 to cause the LED 23 to emit light.

The endoscope 21 is configured such that the insertion portion 22 is inserted into a subject, to thereby be capable of picking up an image of the subject. The endoscope 21 includes an image pickup portion including the LED 23 and an image pickup device 24.

The LED 23 is provided at the insertion portion 22 of the endoscope 21 and configured to be capable of applying illumination light to the subject under the control of the light source driving section 11.

The image pickup device 24 is provided in the insertion portion 22 of the endoscope 21, and arranged so as to be capable of taking in reflection light from the subject irradiated with the illumination light, through an observation window, not shown.

The image pickup device 24 photoelectrically converts the reflection light from the subject, which has been taken in through the observation window, into an image pickup signal, converts the image pickup signal from the analog image pickup signal to a digital image pickup signal by the A/D converter, not shown, and outputs the digital image pickup signal to the control section 32.

The control section 32 transmits a control signal to the light source driving section 11, to be capable of driving the LED 23.

The control section 32 performs image adjustments, for example, gain adjustment, white balance adjustment, gamma correction, contour enhancement correction, and magnification/reduction adjustment, on the image pickup signal inputted from the endoscope 21, to sequentially output observation images G1 of the subject, to be described later, to the detection support section 33.

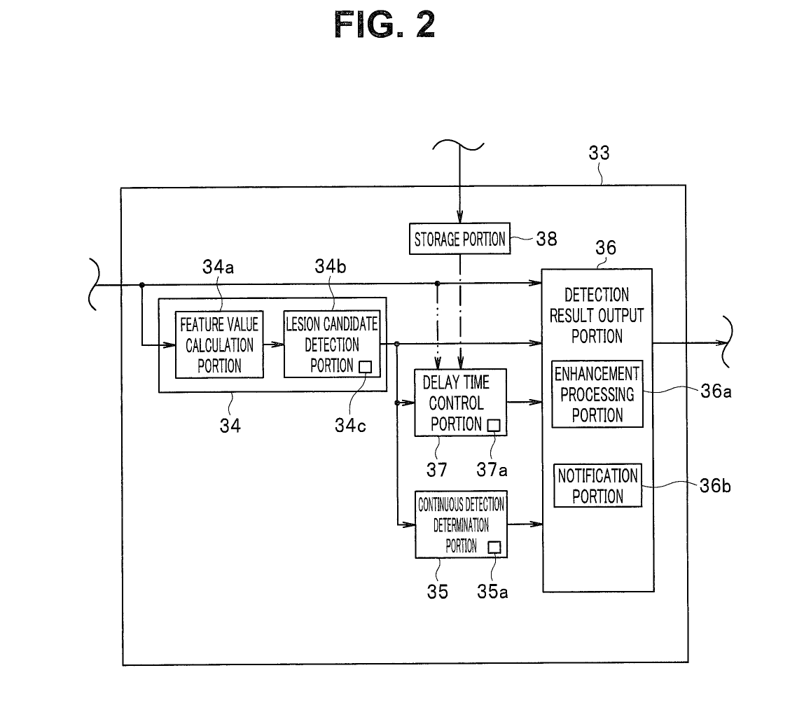

FIG. 2 is a block diagram showing a configuration of the detection support section of the endoscope system according to the embodiment of the present invention. The detection support section 33 is configured to include a function as an endoscopic image processing apparatus. Specifically, as shown in FIG. 2, the detection support section 33 includes a detection portion 34, a continuous detection determination portion 35 as a determination portion, a detection result output portion 36, a delay time control portion 37, and a storage portion 38.

The detection portion 34 is a circuit that sequentially receives the observation images G1 of the subject, to detect a lesion candidate region L as a region-of-interest in each of the observation images G1, based on a predetermined feature value of each of the observation images G1. The detection portion 34 includes a feature value calculation portion 34a and a lesion candidate detection portion 34b.

The feature value calculation portion 34a is a circuit configured to calculate a predetermined feature value of each of the observation images G1 of the subject. The feature value calculation portion 34a is connected to the control section 32 and the lesion candidate detection portion 34b. The feature value calculation portion 34a calculates the predetermined feature value from each of observation images G1 of the subject that are inputted sequentially from the control section 32, and is capable of outputting the calculated feature values to the lesion candidate detection portion 34b.

The predetermined feature value is acquired by calculating, for each predetermined small region on each observation image G1, an amount of change, that is, a tilt value of the respective pixels in the predetermined small region with respect to pixels adjacent to the respective pixels. Note that the method of calculating the feature value is not limited to the method of calculating the feature value based on the tilt value of the respective pixels with respect to the adjacent pixels, but the feature value may be acquired by converting the observation image G1 into a numerical value using another method.

The lesion candidate detection portion 34b is a circuit configured to detect the lesion candidate region L of the observation image G1 from the information on the feature value. The lesion candidate detection portion 34b includes a ROM 34c so as to be capable of storing, in advance, a plurality of pieces of polyp model information. The lesion candidate detection portion 34b is connected to the detection result output portion 36, the continuous detection determination portion 35, and the delay time control portion 37.

The polyp model information includes a feature value of the feature which is common in many polyp images.

The lesion candidate detection portion 34b detects the lesion candidate region L, based on the predetermined feature value inputted from the feature value calculation portion 34a, and a plurality of pieces of polyp model information, and outputs lesion candidate information to the detection result output portion 36, the continuous detection determination portion 35, and the delay time control portion 37.

More specifically, the lesion candidate detection portion 34b compares the predetermined feature value of each of predetermined small regions, which are inputted from the feature value detection portion, with the feature value in the polyp model information stored in the ROM 34c, and when the features are coincident with each other, the lesion candidate detection portion 34b detects the lesion candidate region L. When the lesion candidate region L is detected, the lesion candidate detection portion 34b outputs the lesion candidate information including position information and size information on the detected lesion candidate region L to the detection result output portion 36, the continuous detection determination portion 35, and the delay time control portion 37.

Note that the position information of the lesion candidate region L indicates the position of the lesion candidate region L in the observation image G1, and the position information is acquired as the positions of the pixels of the lesion candidate region L existing in the observation image G1, for example. In addition, the size information of the lesion candidate region L indicates the size of the lesion candidate region L in the observation image G1, and the size information is acquired as the number of pixels of the lesion candidate region L existing in the observation image G1, for example.

Note that the detection portion 34 is not necessarily required to include the feature value calculation portion 34a and the lesion candidate detection portion 34b, as long as the detection portion 34 is configured to perform the processing for detecting the lesion candidate region L from each of the observation images G1. Specifically, the detection portion 34 may be configured to detect the lesion candidate region L from each of the observation images G1 by performing processing on each of the observation images G1 with an image discriminator that acquires, in advance, a function for discriminating a polyp image using a learning method such as deep learning.

The continuous detection determination portion 35 is a circuit configured to determine whether or not the lesion candidate region L is detected continuously. The continuous detection determination portion 35 includes a RAM 35a so as to be capable of storing the lesion candidate information in at least one frame before the current frame. The continuous detection determination portion 35 is connected to the detection result output portion 36.

The continuous detection determination portion 35 determines whether or not a first lesion candidate region on a first observation image and a second lesion candidate region on a second observation image inputted earlier than the first observation image are the same lesion candidate region L, so that the lesion candidate region L can be tracked even when the position of the lesion candidate region L is shifted on the observation images G1, for example, and when the same lesion candidate region L is continuously or intermittently detected on a plurality of observation images G1 that are inputted sequentially, the continuous detection determination portion 35 determines that the lesion candidate region L is continuously detected, and outputs the determination result to the detection result output portion 36.

The detection result output portion 36 is a circuit configured to perform output processing of the detection result. The detection result output portion 36 includes an enhancement processing portion 36a and a notification portion 36b. The detection result output portion 36 is connected to the display section 41. The detection result output portion 36 is capable of perfo1ining enhancement processing and notification processing, based on the observation images G1 inputted from the control section 32, the lesion candidate information inputted from the lesion candidate detection portion 34b, the determination result inputted from the continuous detection determination portion 35, and a first period of time (to be described later, and hereinafter just referred to as first period) controlled by the delay time control portion 37. The detection result output portion 36 outputs the image for display G to the display section 41.

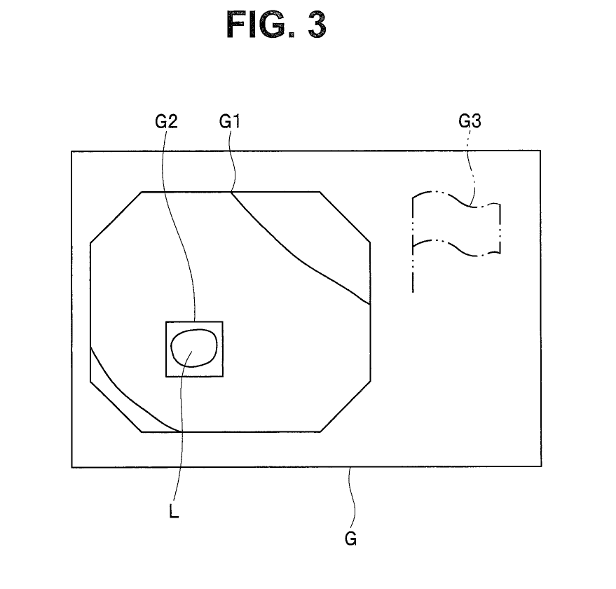

FIG. 3 is an explanatory diagram illustrative of an example of a screen configuration of an image for display of the endoscope system according to the embodiment of the present invention. As shown in FIG. 3, the observation image G1 is arranged in the image for display G outputted from the detection result output portion 36. FIG. 3 illustrates the internal wall of the large intestine including the lesion candidate region L, as one example of the observation image G1.

When the lesion candidate region L is continuously detected by the lesion candidate detection portion 34b, the enhancement processing portion 36a performs enhancement processing of the position corresponding to the legion candidate region L for the observation image G1 of the subject which is inputted after an elapse of the first period from the time point of the starting of the detection of the lesion candidate region L. That is, the enhancement processing is started when the lesion candidate region L, which is determined to be continuously detected by the continuous detection determination portion 35, is detected continuously for the first period.

The enhancement processing is performed for a second period of time (hereinafter, just referred to as second period) at the longest, and is ended after the elapse of the second period. If the continuous detection of the lesion candidate region L by the continuous detection determination portion 35 is ended before the elapse of the second period, also the enhancement processing is ended at that time.

More specifically, in the case where the enhancement processing is started after the elapse of the first period and thereafter the second period further elapses, even if the lesion candidate region L, which is determined to be continuously detected by the continuous detection determination portion 35, is continuously detected, the enhancement processing is ended.

The second period is a predetermined time for which the operator is capable of sufficiently recognize the lesion candidate region L from a marker image G2, and is set, in advance, to 1.5 seconds, for example. In addition, the second period is defined depending on the number of frames. Specifically, when the number of frames per second is 30, for example, the second period is defined as 45 frames.

The enhancement processing is processing for performing a display showing the position of the lesion candidate region L. More specifically, the enhancement processing is processing for adding a marker image G2 that surrounds the lesion candidate region L to the observation image G1 inputted from the control section 32, based on the position information and the size information included in the lesion candidate information. Note that the marker image G2 is shown in a square shape in FIG. 3, as one example. However, the marker image may have any shape such as triangle, circle, star, etc.

The notification portion 36b is configured to be capable of notifying the operator of the existence of the lesion candidate region L in the observation image G1, by the notification processing different from the enhancement processing. The notification processing is performed during a period after the elapse of the second period when the enhancement processing is ended until the continuous detection of the lesion candidate region L by the detection portion 34 is ended.

The notification processing is processing for adding a notification image G3 to a region outside the observation image G1 in the image for display G. In FIG. 3, the notification image G3 is illustrated as a flag pattern with the two-dot-chain lines, as one example. However, the notification image G3 may have any shape such as triangle, circle, star, etc.

The delay time control portion 37 includes an arithmetic circuit etc., for example Moreover, the delay time control portion 37 includes a RAM 37a that is capable of storing the lesion candidate information of at least one frame before the current frame. In addition, the delay time control portion 37 is connected to the detection result output portion 36.

The delay time control portion 37 performs, on the detection result output portion 36, control for setting an initial value (also referred to as initially set time) of the first period which is delay time after the lesion candidate region L is detected until the enhancement processing is started. In addition, the delay time control portion 37 is configured to be capable of performing, on the detection result output portion 36, control for changing the first period within a range that is longer than zero second and shorter than the second period, based on the position information and the size information included in the lesion candidate information inputted from the lesion candidate detection portion 34b. The initial value of the first period is predetermined time, and is set, in advance, to 0.5 seconds, for example. In addition, the first period is defined by the number of frames. Specifically, when the number of frames per second is 30, the first period is defined as 15 frames, for example.

The storage portion 38 includes a storage circuit such as a memory. In addition, the storage portion 38 is configured to store operator information indicating the proficiency and/or the experienced number of examinations of the operator who actually observes the subject using the endoscope 21, when the operator information is inputted by operating the input device 51.

The display section 41 is configured by a monitor, and capable of displaying the image for display G, which is inputted from the detection result output portion 36, on the screen.

The input device 51 includes a user interface such as a key board, and is configured to be capable of inputting various kinds of information to the detection support section 33. Specifically, the input device 51 is configured to be capable of inputting the operator information in accordance with the operation by the user to the detection support section 33, for example.

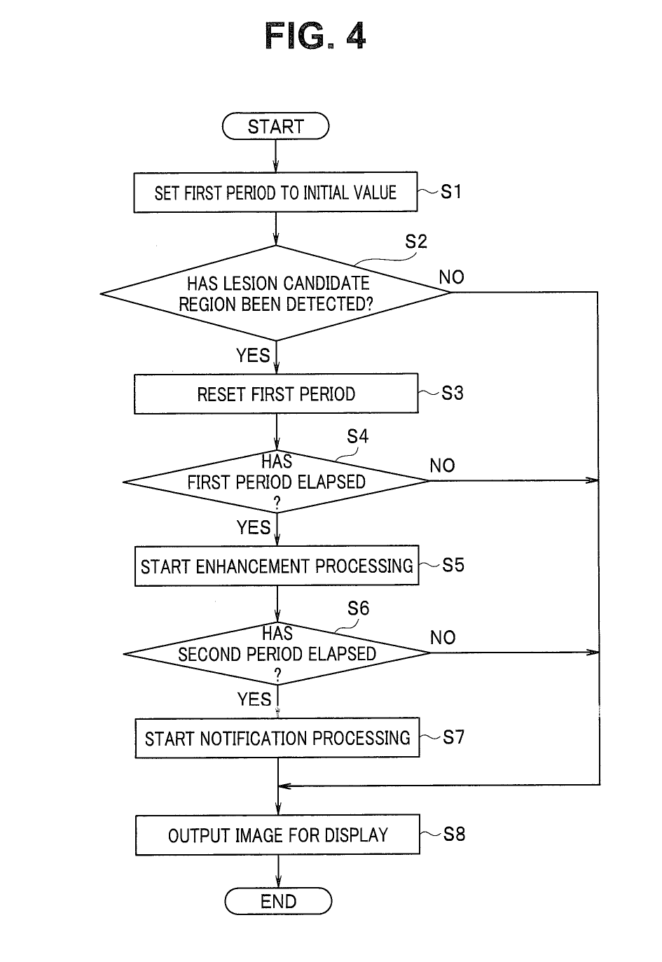

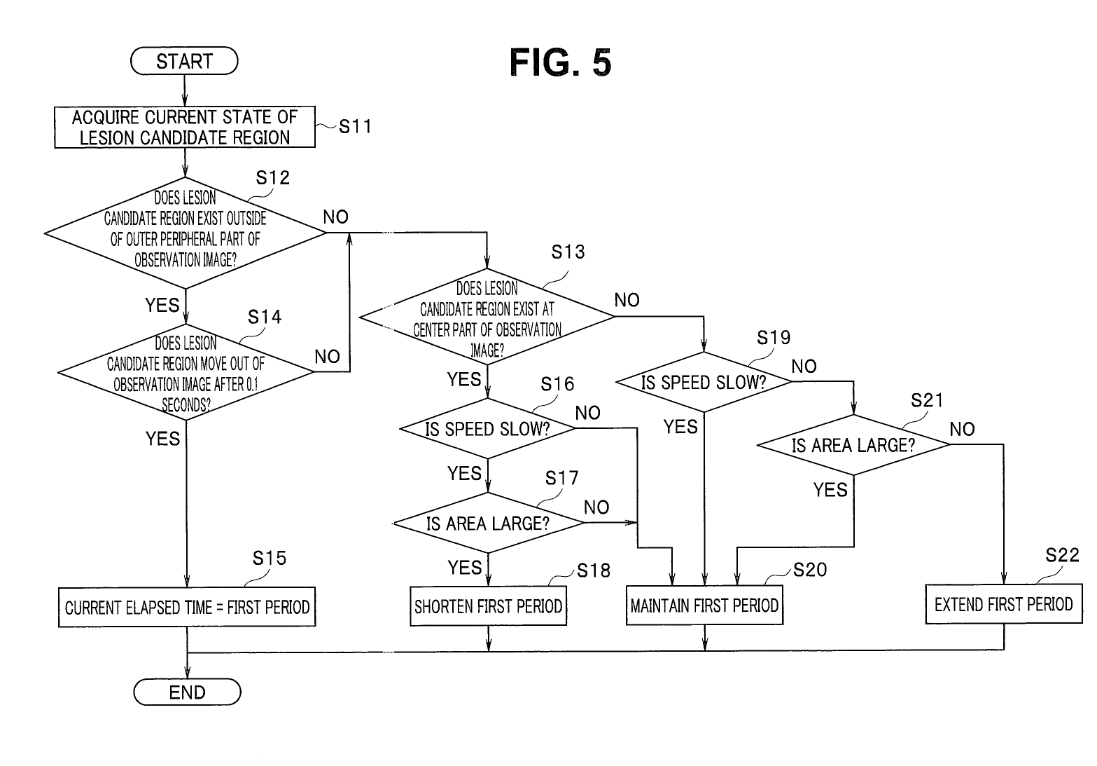

Next, specific examples of the processing performed in the detection result output portion 36 and the delay time control portion 37 in the endoscope system 1 according to the embodiment will be described referring to FIGS. 4 and 5. Each of FIGS. 4 and 5 is a flowchart describing one example of the processing performed in the endoscope system according to the embodiment of the present invention.

When the image of the subject is picked up by the endoscope 21, the image adjusting processing is performed by the control section 32, and thereafter the observation image G1 is inputted to the detection support section 33. When the observation image G1 is inputted to the detection support section 33, the feature value calculation portion 34a calculates a predetermined feature value of the observation image G1, to output the calculated feature value to the lesion candidate detection portion 34b. The lesion candidate detection portion 34b compares the inputted predetermined feature value with the feature value in the polyp model information, to detect the lesion candidate region L. The detection result of the lesion candidate region L is outputted to the continuous detection determination portion 35, the detection result output portion 36, and delay time control portion 37. The continuous detection determination portion 35 determines whether or not the lesion candidate region L is continuously detected, to output the determination result to the detection result output portion 36.

Based on the detection result of the lesion candidate region L inputted from the lesion candidate detection portion 34b, the delay time control portion 37 performs, on the detection result output portion 36, control for setting the initial value of the first period in a period during which the lesion candidate region L is not detected, for example. The detection result output portion 36 sets the initial value of the first period according to the control by the delay time control portion 37 (S1).

The detection result output portion 36 determines whether or not the lesion candidate region L has been detected based on the detection result of the lesion candidate region L inputted from the lesion candidate detection portion 34b (S2).

When acquiring the determination result that the lesion candidate region L has been detected (S2: Yes), the detection result output portion 36 starts to measure the elapsed time after the lesion candidate region L has been detected, and resets the first period according to the control by the delay time control portion 37. In addition, when acquiring the determination result that the lesion candidate region L is not detected (S2: No), the detection result output portion 36 performs processing for outputting the image for display G to the display section 41 (S8).

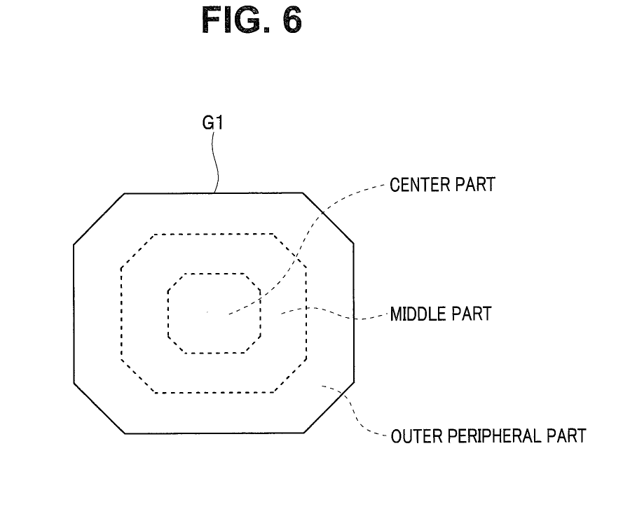

Hereinafter, a specific example of the control related to the resetting of the first period by the delay time control portion 37 will be described referring to FIGS. 5 and 6. FIG. 6 is a schematic diagram showing one example of a classifying method of respective parts of the observation image to be used in the processing in FIG. 5.

The delay time control portion 37 performs processing for acquiring the current state of the lesion candidate region L, based on the lesion candidate information inputted from the lesion candidate detection portion 34b and the lesion candidate information stored in the RAM 37a (S11). Specifically, the delay time control portion 37 acquires the current position of the center of the lesion candidate region L, based on the position information included in the lesion candidate information inputted from the lesion candidate detection portion 34b. In addition, the delay time control portion 37 acquires the current moving speed and moving direction of the center of the lesion candidate region L, based on the position information included in the lesion candidate information, which is inputted from the lesion candidate detection portion 34b, and the position information in the one frame before the current frame, which is included in the lesion candidate information stored in the RAM 37a. Furthermore, the delay time control portion 37 acquires the area of the lesion candidate region L, based on the size information included in the lesion candidate information inputted from the lesion candidate detection portion 34b.

The delay time control portion 37 determines whether or not the lesion candidate region L exists in an outer peripheral part (see FIG. 6) of the observation image G1, based on the current position of the center of the lesion candidate region L, which has been acquired by the processing in S11 (S12).

When acquiring the determination result that the lesion candidate region L exists in the outer peripheral part of the observation image G1 (S12: Yes), the delay time control portion 37 performs the processing in S14 to be described later. Moreover, when acquiring the determination result that the lesion candidate region L does not exist in the outer peripheral part of the observation image G1 (S12: No), the delay time control portion 37 determines whether or not the lesion candidate region L exists in the center part (See FIG. 6) of the observation image G1, based on the current position of the center of the lesion candidate region L, which has been acquired by the processing in S11 (S13).

When acquiring the determination result that the lesion candidate region L exists in the center part of the observation image G1 (S13: Yes), the delay time control portion 37 performs processing in S16 to be described later. Moreover, when acquiring the determination result that the lesion candidate region L does not exist in the center part of the observation image G1 (S13: No), the delay time control portion 37 performs processing in S19 to be described later.

That is, in the processing in S12 and S13, when the lesion candidate region L exists neither in the outer peripheral part nor in the center part of the observation image G1 (S12: No and S13: No), the subsequent processing is performed supposing that the lesion candidate region L exists in the middle part (see FIG. 6) of the observation image G1.

The delay time control portion 37 determines whether or not the lesion candidate region L moves out of the observation image G1 after 0.1 seconds, based on the current moving speed and moving direction of center of the lesion candidate region L, which have been acquired by the processing in S1l (S14).

When acquiring the determination result that the lesion candidate region L moves out of the observation image G1 after 0.1 seconds (S14: Yes), the delay time control portion 37 performs processing in S15 to be described later. Furthennore, when acquiring a determination result that the lesion candidate region L does not move out of the observation image G1 after 0.1 seconds (S14: No), the delay time control portion 37 performs the processing in S13 as described above.

The delay time control portion 37 performs, on the detection result output portion 36, control for resetting, as the first period, the current elapsed time elapsed from the time point of the staring of the detection of the lesion candidate region L (S15).

The delay time control portion 37 determines whether or not the moving speed of the lesion candidate region L is slow, based on the current moving speed of the center of the lesion candidate region L, which has been acquired in the processing in S11 (S16). Specifically, the delay time control portion 37 acquires a determination result that the moving speed of the lesion candidate region L is slow, when the current moving speed of the center of the lesion candidate region L, which has been acquired in the processing in S11, is 50 pixels per second or less, for example. Moreover, the delay time control portion 37 acquires a determination result that the moving speed of the lesion candidate region L is fast, when the current moving speed of the center of the lesion candidate region L, which has been acquired by the processing in S11, exceeds 50 pixels per second, for example.

When acquiring the determination result that the moving speed of the lesion candidate region L is slow (S16: Yes), the delay time control portion 37 performs processing in S17 to be described later. Furthermore, when acquiring the determination result that the moving speed of the lesion candidate region L is fast (S16: No), the delay time control portion 37 performs processing in S20 to be described later.

The delay time control portion 37 determines whether or not the area of the lesion candidate region L is large, based on the area of the lesion candidate region L, which has been acquired by the processing in S11 (S17). Specifically, the delay time control portion 37 acquires the determination result that the area of the lesion candidate region L is large, when the ratio of the area (the number of pixels) of the lesion candidate region L, which has been acquired by the processing in S11, to the total area (the total number of pixels) of the observation image G1 is 5% or larger, for example. Furthermore, the delay time control portion 37 acquires the determination result that the area of the lesion candidate region L is small, when the ratio of the area (the number of pixels) of the lesion candidate region L, which has been acquired by the processing in S11, to the total area (the total number of pixels) of the observation image G1 is smaller than 5%, for example.

When acquiring the determination result that the area of the lesion candidate region L is large (S17: Yes), the delay time control portion 37 performs processing in S18 to be described later. Furthermore, when acquiring the determination result that the area of the lesion candidate region L is small (S17: No), the delay time control portion 37 performs the processing in S20 to be described later.

The delay time control portion 37 performs, on the detection result output portion 36, control for resetting the first period to the time shorter than the initially set time (initial value), that is, control for shortening the first period to less than the initial value (S18).

The delay time control portion 37 determines whether or not the moving speed of the lesion candidate region L is slow, based on the current moving speed of the center of the lesion candidate region L, which has been acquired by the processing in S1l (S19). Specifically, the delay time control portion 37 performs the processing same as that in S16, for example, to thereby acquire either the determination result that the moving speed of the lesion candidate region L is slow, or the determination result that the moving speed of the lesion candidate region L is fast.

When acquiring the determination result that the moving speed of the lesion candidate region L is slow (S19: Yes), the delay time control portion 37 performs processing in S20, to be described later. Furthermore, when acquiring the determination result that the moving speed of the lesion candidate region L is fast (S19: No), the delay time control portion 37 performs processing in S21 to be described later.

The delay time control portion 37 performs, on the detection result output portion 36, the control for resetting the first period to the time equal to the initially set time, i.e., the control for maintaining the first period at the initial value (S20).

The delay time control portion 37 determines whether or not the area of the lesion candidate region L is large, based on the area of the lesion candidate region L, which has been acquired by the processing in S11 (S21). Specifically, the delay time control portion 37 performs the processing same as that in S17, for example, to thereby acquire either the determination result that the area of the lesion candidate region L is large, or the determination result that the area of the lesion candidate region L is small.

When acquiring the determination result that the area of the lesion candidate region L is large (S21: Yes), the delay time control portion 37 performs the processing in S20 as described above. Furthermore, when acquiring the determination result that the area of the lesion candidate region L is small (S21: No), the delay time control portion 37 performs processing in S22 to be described later.

The delay time control portion 37 performs, on the detection result output portion 36, the control for resetting the first period to the time longer than the initially set time, i.e., the control for extending the first period to more than the initial value (S22).

With the processing in S11 to S13 and S16 to S22 as described above, the delay time control portion 37 determines whether or not the visibility of the lesion candidate region L in the observation image G1 is high, based on the position information and the size information included in the lesion candidate information inputted from the lesion candidate detection portion 34b, to acquire a determination result. Then, based on the acquired determination result, when the visibility of the lesion candidate region L in the observation image G1 is high, the delay time control portion 37 resets the first period to the time shorter than the initially set time, and when the visibility of the lesion candidate region L in the observation image G1 is low, the delay time control portion 37 resets the first period to the time longer than the initially set time. In addition, with the processing in S11, S12, S14, and S15 as described above, the delay time control portion 37 determines whether or not disappearance possibility of the lesion candidate region L from inside of the observation image G1 is high, based on the position information included in the lesion candidate information inputted from the lesion candidate detection portion 34b, to acquire a determination result. Then, based on the acquired determination result, when the disappearance possibility of the lesion candidate region L from inside of the observation image G1 is high, the delay time control portion 37 causes the enhancement processing to start immediately at the current elapsed time elapsed from the time point of the starting of the detection of the lesion candidate region L.

Note that the delay time control portion 37 according to the present embodiment may determine whether or not the visibility of the lesion candidate region L in the observation image G1 is high, based on the position information and the size information included in the lesion candidate information inputted from the lesion candidate detection portion 34b and the proficiency and/or the experienced number of examinations included in the operator information stored in the storage portion 38, for example, to acquire a determination result (one-dot-chain line in FIG. 2). Then, in such a configuration, when the proficiency of the operator, which is included in the operator information stored in the storage portion 38, is high and/or the experienced number of examinations included in the operator information stored in the storage portion 38 is large, for example, the delay time control portion 37 may shorten the first period to less than the initial value (or maintain the first period at the initial value).

In addition, the delay time control portion 37 according to the present embodiment may determine whether or not the visibility of the lesion candidate region L in the observation image G1 is high, based on the position information and the size information included in the lesion candidate information inputted from the lesion candidate detection portion 34b, and a predetermined parameter indicating the clearness of the lesion candidate region L included in the observation image G1 inputted from the control section 32, for example, to acquire a determination result (see two-dot-chain line in FIG. 2). In such a configuration, when the contrast, the saturation, the luminance, and/or the sharpness of the observation image G1 inputted from the control section 32 are high, for example, the delay time control portion 37 may shorten the first period to less than the initial value (or maintain the first period at the initial value).

In addition, the delay time control portion 37 according to the present embodiment is not limited to the configuration in which the first period is reset based on both the position information and the size information included in the lesion candidate information inputted from the lesion candidate detection portion 34b, but the delay time control portion 37 may be configured to reset the first period based on one of the position information and the size infotination, for example.

In addition, the delay time control portion 37 according to the present embodiment is not limited to the configuration in which the first period is reset based on both the determination result acquired by determining whether or not the visibility of the lesion candidate region L in the observation image G1 is high and the determination result acquired by determining whether or not the disappearance possibility of the lesion candidate region L from inside of the observation image G1 is high, but the delay time control portion 37 may be configured to reset the first period using one of the above-described detennination results, for example.

The detection result output portion 36 determines whether or not the first period reset by the processing in S3 has elapsed after the detection of the lesion candidate region L (S4).

When the first period reset by the processing in S3 has elapsed after the detection of the lesion candidate region L (S4: Yes), the detection result output portion 36 starts the enhancement processing for adding the marker image G2 to the observation image G1 (S5). Moreover, when the first period reset by the processing in S3 has not elapsed after the detection of the lesion candidate region L (S4: No), the detection result output portion 36 performs processing for outputting the image for display G to the display section 41 (S8).

The detection result output portion 36 determines whether or not the second period has elapsed after performing the processing in S5 (S6).

When the second period has elapsed after performing the processing in S5 (S6: Yes), the detection result output portion 36 removes the marker image G2 from the observation image G1 to end the enhancement processing, and starts the notification processing for adding the notification image G3 to the region outside the observation image G1 in the image for display G (S7). In addition, when the second period has not elapsed after performing the processing in S5 (S6: No), the detection result output portion 36 performs processing for outputting the image for display G to the display section 41 (S8). That is, the detection result output portion 36 ends the enhancement processing when the second period has further elapsed after the elapse of the first period reset by the processing in S3.

Note that, for explanation, the number of the lesion candidate region L displayed on the observation screen is one in the present embodiment, but there is a case where a plurality of lesion candidate regions L are displayed on the observation screen. In that case, the enhancement processing is performed on the plurality of lesion candidate regions L. The enhancement processing of the respective lesion candidate regions L is performed on the observation image G1 inputted when the first period elapses after the detection of the respective lesion candidate regions L.

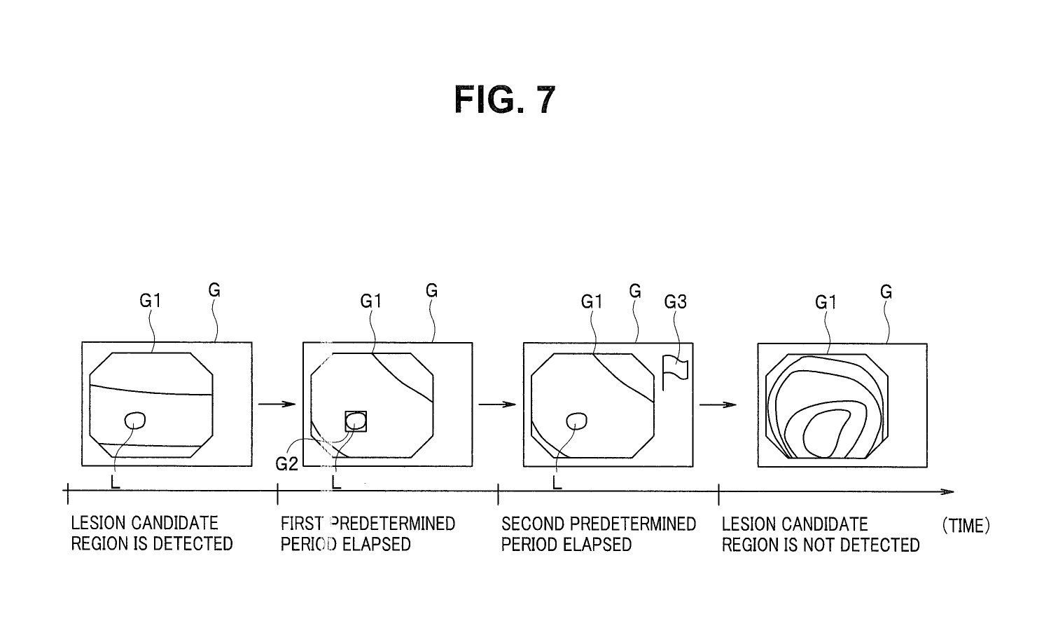

The above-described processing in S1 to S8 and S11 to S22 is repeatedly perfoimed, which causes the display state of the image for display G to transit as shown in FIG. 7, for example. FIG. 7 is a view illustrating one example of a screen transition of the image for display in accordance with the processing performed in the endoscope system according to the embodiment of the present invention.

First, the marker image G2 is not displayed until the first period elapses after the first detection of the lesion candidate region L. Subsequently, when the lesion candidate region L is detected continuously for the first period, the enhancement processing portion 36a starts the enhancement processing, and the marker image G2 is displayed in the image for display G. Next, when the lesion candidate region L is detected continuously even after the elapse of the second period, the enhancement processing is ended, and the notification processing is started by the notification portion 36b. Then, the marker image G2 is brought into the non-display state and the notification image G3 is displayed in the image for display G. Then, when the lesion candidate region L is no longer detected, the notification processing is ended and the notification image G3 is brought into the non-display state.

As described above, according to the present embodiment, when a plurality of lesion candidate regions L exist in an observation image G1 and the lesion candidate region L which is likely to move out of the observation image G1 exists in the observation image G1, for example, the first period is shortened to less than the initial value, thereby being capable of preventing the overlooking of the lesion part by the operator's visual confirmation as much as possible. In addition, according to the present embodiment, for example, the lesion candidate region L, the size of which is small and the moving speed of which is fast exists in the outer peripheral part of the observation image G1, the first period is extended more than the initial value, thereby being capable of preventing the oversight of the lesion part by the operator's visual confirmation as much as possible. That is, the present embodiment suppresses the decline of the operator's attentiveness to the observation image G1, and is capable of presenting a region-of-interest without interfering with the improvement of the lesion part finding performance.

Note that, in the present embodiment, the control section 32 performs image adjustments, for example, gain adjustment, white balance adjustment, gamma correction, contour enhancement correction, and magnification/reduction adjustment, on the image pickup signal inputted from the endoscope 21, to input the observation image G1 subjected to the image adjustments to the detection support section 33. However, all of or a part of the image adjustments may be performed on the image signal outputted from the detection support section 33, instead of the image signal before being inputted to the detection support section 33.

In addition, in the present embodiment, the enhancement processing portion 36a adds the marker image G2 to the lesion candidate region L, but the marker image G2 may be displayed by being classified by color depending on the degree of certainty of the detected lesion candidate region L. In this case, the lesion candidate detection portion 34b outputs the lesion candidate information including the infoiniation on the degree of certainty of the lesion candidate region L to the enhancement processing portion 36a, and the enhancement processing portion 36a performs enhancement processing according to the color classification based on the degree of certainty of the lesion candidate region L. According to such a configuration, when observing the lesion candidate region L, the operator can estimate that the possibility of the false positive (erroneous detection) is high or low based on the color of the marker image G2.

In addition, according to the present embodiment, the detection support section 33 is configured by a circuit, but the respective functions of the detection support section 33 may be configured by a processing program, the function of which is implemented by the processing by the CPU.

The image processing apparatus and the like according to the present embodiment may include a processor and a storage (e.g., a memory). The functions of individual units in the processor may be implemented by respective pieces of hardware or may be implemented by an integrated piece of hardware, for example. The processor may include hardware, and the hardware may include at least one of a circuit for processing digital signals and a circuit for processing analog signals, for example. The processor may include one or a plurality of circuit devices (e.g., an IC) or one or a plurality of circuit elements (e.g., a resistor, a capacitor) on a circuit board, for example. The processor may be a CPU (Central Processing Unit), for example, but this should not be construed in a limiting sense, and various types of processors including a GPU (Graphics Processing Unit) and a DSP (Digital Signal Processor) may be used. The processor may be a hardware circuit with an ASIC. The processor may include an amplification circuit, a filter circuit, or the like for processing analog signals. The memory may be a semiconductor memory such as an SRAM and a DRAM; a register; a magnetic storage device such as a hard disk device; and an optical storage device such as an optical disk device. The memory stores computer-readable instructions, for example. When the instructions are executed by the processor, the functions of each unit of the image processing device and the like are implemented. The instructions may be a set of instructions constituting a program or an instruction for causing an operation on the hardware circuit of the processor.

The units in the image processing apparatus and the like and the display device according to the present embodiment may be connected with each other via any types of digital data communication such as a communication network or via communication media. The communication network may include a LAN (Local Area Network), a WAN (Wide Area Network), and computers and networks which form the interne, for example.

What is claimed is:

1. An endoscopic image processing apparatus comprising a processor, the processor being configured to:
perform processing for detecting a region-of-interest from sequentially inputted observation images of a subject;
perform enhancement processing of a position corresponding to the region-of-interest, on the observation images of the subject inputted after a first period elapses from a time point of a start of detection of the region-of-interest, when the region-of-interest is continuously detected; and
set the first period based on at least one of position information indicating a position of the region-of-interest in the observation images and size information indicating a size of the region-of-interest in the observation images.

2. The endoscopic image processing apparatus according to claim 1, wherein the processor sets the first period based on at least one of a determination result acquired by determining whether or not visibility of the region-of-interest in the observation images is high based on at least one of the position information and the size information, and a determination result acquired by determining whether or not a disappearance possibility of the region-of-interest from inside of the observation images is high based on the position information.

3. The endoscopic image processing apparatus according to claim 2, wherein the processor determines whether or not the visibility of the region-of-interest in the observation images is high, based on the position and a moving speed of the region-of-interest, which are acquired from the position information, and a ratio of an area of the region-of-interest to a total area of each of the observation images, which is acquired from the size information.

4. The endoscopic image processing apparatus according to claim 3, wherein the processor further determines whether or not the visibility of the region-of-interest in the observation images is high, based on proficiency and/or an experienced number of examinations of an operator who actually observes the subject.

5. The endoscopic image processing apparatus according to claim 3, wherein the processor further determines whether or not the visibility of the region-of-interest in the observation image is high, based on a predetermined parameter indicating clearness of the region-of-interest.

6. The endoscopic image processing apparatus according to claim 3, wherein the processor sets the first period to a time shorter than a predetermined time when the visibility of the region-of-interest in the observation images is high, and sets the first period to a time longer than the predetermined time when the visibility of the region-of-interest in the observation images is low.

7. The endoscopic image processing apparatus according to claim 2, wherein the processor determines whether or not the disappearance possibility of the region-of-interest from inside of the observation images is high, based on the position of the region-of-interest, a moving speed of the region-of-interest, and a moving direction of the region-of-interest, which are acquired from the position information.

8. The endoscopic image processing apparatus according to claim 7, wherein the processor sets a current elapsed time elapsed from the time point of the start of detection of the region-of-interest, as the first period, when the disappearance possibility of the region-of-interest from inside of the observation images is high.

9. The endoscopic image processing apparatus according to claim 1, wherein the processor ends the enhancement processing when a second period further elapses after an elapse of the first period.
